(19) United States
(12) Plant Patent Application Publication
(10) Pub. No.: US 2007/0283463 P1
(43) Pub. Date: Dec. 6, 2007

(54) Climbing rose plant named 'Poulyc001'

(76) Inventor: Mogens Olesen, Fredensborg (DK)

Correspondence Address: POULSEN ROSER APS, 620 S. FRONT STREET, CENTRAL POINT, OR, 97502, US

(21) Appl. No.: 11/891,113

(22) Filed: Aug. 8, 2007

Related U.S. Application Data
(63) Continuation of application No. 10/342,688, filed on Jan. 14, 2003, now abandoned.
(63) Continuation of application No. 11/090,135, filed on Mar. 24, 2005.

(51) Int. Cl. A01H 5/00

(52) U.S. Cl. PLT/115

(57) ABSTRACT

A new climbing garden rose plant with abundant, light pink flowers and attractive foliage. This new and distinct variety has shown to be uniform and stable in the resulting generations from asexual propagation.

### BOTANICAL DESIGNATION

Rosa hybrida.

### VARIETY DENOMINATION

‘Poulyc001.’

### SUMMARY OF THE INVENTION

The present invention constitutes a new and distinct variety of garden rose plant which originated from a controlled crossing between an unnamed, non-patented seedling and ‘Meidomonac’ Bonica 82. The two parents were crossed during the-summer of 1991 and the resulting seeds were planted in a controlled environment in Fredensborg, Denmark. The new variety is named ‘Poulyc001’

The new variety may be distinguished from its seed parent, ‘Meidomonac’ Bonica 82, by the following combination of characteristics:
1. The pollen parent has a low growing shrub habit, whereas ‘Poulyc001’ has a broad, climbing growth habit.
2. The pollen parent has 20 flower buds per stem, whereas ‘POULcy001’ has 5 to 7 flower buds per stem.

The objective of the hybridization of this rose variety was to create a new and distinct variety for garden use with unique qualities, such as:
1. Uniform and abundant flowers;
2. Vigorous, but compact growth when propagated both as a budded rose and on its own roots;
3. Disease resistance.

This combination of qualities is not present in previously available commercial cultivars of this type and distinguish ‘Poulyc001’ from all other varieties of which we are aware.

As part of the rose development program, Mogens N. Olesen germinated the seeds from the aforementioned hybridization during winter 1991 and conducted evaluations on the resulting seedlings in a controlled environment in Fredensborg, Denmark.

‘Poulyc001’ was selected in the spring 1992 by the inventor as a single plant from the progeny of the aforementioned hybridization.

Asexual reproduction of ‘Poulyc001’ by traditional budding and rooted cuttings was first done by Mogens N. Olesen in the nursery in Fredensborg, Denmark in August, 1992. This initial and other subsequent asexual propagations conducted in controlled environments have demonstrated that the characteristics of ‘Poulyc001’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems, of ‘Poulyc001’. Specifically illustrated in SHEET 1:

FIG. 1.1 Flower bud, partially opened bud, and open bloom;

FIG. 1.2 Flower petals, detached;

FIG. 1.3 Stem showing cluster of open flowers, branching and the attachment of leaves, buds, and peduncles;

FIG. 1.4 Sepals, receptacle, and pedicel;

FIG. 1.5 Bare stems;

FIG. 1.6 Leaves.

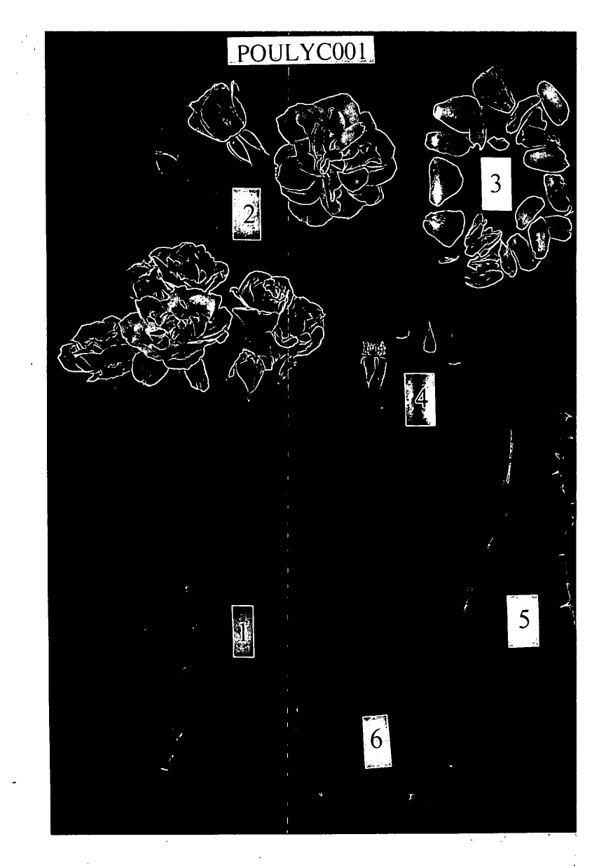

### DETAILED DESCRIPTION OF THE VARIETY

The following is a description of ‘Poulyc001’, as observed in its growth in a field nursery in Jackson County, Oregon. Color references are made using The Royal Horticultural Society (London, England) Color Chart, 1995, except where common terms of color are used.

For a comparison, several physical characteristics of the rose variety ‘Meidomonac’, a rose variety described and illustrated in U.S. Plant Pat. No. 5,105 issued Sep. 20, 1983 are compared to ‘Poulyc001’ in Chart 1.

CHART 1

| | ‘Poulyc001’ | ′Meidomonac′ |
|---|---|---|
| Growth habit | Climbing | Low Growing Shrub |
| Flower bud | 3 cm from base of | 1.4 cm from base of |
| size | receptacle to end | receptacle to end |
| | of bud | of bud |
| Flowers per | 5 to 7 | 20 |
| stem | | |

Parents:
   Seed parent.—‘Meidomonac’ Bonica 82.
   Pollen parent.—An unnamed seedling.

### FLOWER AND FLOWER BUD

Blooming habit: Continuous.
Flower bud:
   Size.—Upon opening, 30 mm in length from base of receptacle to end of bud.
   Bud form.—Pointed ovoid.
   Bud color.—As sepals unfold, petals are Red Group 55B to 55C. At ¼ opening petals are Red Group 55B to 55C.
   Sepals.—Yellow-Green Group 144A. Medium foliaceous appendages on three of the five sepals. Surfaces of sepals are moderately to abundantly pubescent. Stipitate glands are present along the margins and lower surfaces of the sepals. Shape: Sepal apex is cirrose. Base is flat at union with peduncle. Size: 20 to 25 mm (l)×6 to 7 mm (w).
   Receptacle.—Surface: Surface is generally characterized by fine pubescence. Shape: Urn shaped. Size: Small, 6 mm (h)×5 mm (w). Color: Yellow-Green Group 144A.
   Peduncle.—Surface: Smooth with occasional stipitate glands. Length: 25 to 35 mm average length. Color: Yellow-Green Group 144B. Strength: Somewhat strong.
   Borne.—5 to 7 buds per stem.
   Anthocyanin.—Color: Greyed-Red Group 181A.
Flower bloom:
   Fragrance.—Light to none.
   Duration.—The blooms have a duration on the plant of approximately 7 to 10 days. Petals fall cleanly away from plant.
   Size.—Average flower diameter is 50 to 60 mm when open.
   Form.—Shape of flower when viewed from the side: Upon opening, upper part: Flattened convex. Upon opening, lower part: Flattened convex. Open flower, upper part: Flattened convex. Open flower, lower part: Flattened convex.
   Petalage.—Semi double. Average range.: 18 to 20 petals under normal conditions with 5 to 8 petaloids.
Color:
   Upon opening, petals.—Outermost petals: Outer side: Red Group 55B to 55C. Inner Side: Red Group 55 C to 55D with basal intonations of Red Group 56C. Innermost petals: Outer side: Red Group 55B to 55C. Inner Side: Red Group 55C to 55D.
   Upon opening, basal petal spots.—Outermost petals: Outer side: Yellow Group 4C. Inner Side: Yellow Group 4B. Innermost petals: Outer side: Yellow Group 4B. Inner Side: Yellow Group 4B.
   After opening, petals.—Outermost petals: Outer side: Red Group 55D with basal intonations of Red Group 56D. Inner Side: Red Group 55D with basal intonations of Red Group 56D. Innermost petals: Outer side: Red Group 55C to 55D. Inner Side: Red Group 55D.
   After opening, basal petal spots.—Outermost petals: Outer Side: Yellow Group 4D. Inner Side: Yellow Group 4C. Innermost petals: Outer Side: Yellow Group 4C. Inner Side: Yellow Group 4C.
General tonality: On open flower Red Group 55C. No change in the general tonality at the end of the 3rd day. Afterwards, general tonality is Red Group 56D.
Petals:
   Petal reflex.—Slightly reflexed.
   Petal edge.—Uniform and typically reflexed along margin, with a point in the center of the margin.
   Shape.—Obovate shaped. Petaloids: Present. Quantity: 5 to 8.
   Thickness.—Average.
   Arrangement.—Not formal.
Reproductive organs:
   Pistils.—Length: 9 mm long. Quantity: 25.
   Pollen.—Color: Yellow-Orange Group 22A. Quantity: Abundant.
   Anthers.—Size: 2 mm long. Color: Yellow-Orange Group 22A at the margins. Red Group 37 A at mid section. Quantity: 45.
   Filaments.—Color: Yellow-Green Group 150C. Length: 6 mm.
   Stigmas.—Superior in location to anthers. Color: Orange-Red Group 35D.
   Styles.—Color: Yellow-Green Group 150C.
   Hips.—Quantity: Abundant. Size: 10 to 12 mm (w) by 12 to 14 mm (l). Color: Yellow-Green Group 146B with intonations of Greyed-Orange 174A. Shape: Spherical.

### PLANT

Plant growth: Vigorous, with long, arching flowering shoots. When grown as a budded field grown plant on Rosa multiflora understock, the average height of the plant is 150 to 200 cm and the average width is 60 cm.
Stems:
   Color.—Young wood: Yellow-Green Group 144B. Older wood: Yellow-Green Group 144B.
   Thorns.—Incidence: 10 to 14 thorns per 10 cm of stem. Size: Average length: 6 to 7 mm. Color: Greyed-Yellow Group 162A. Shape: Deeply concave.
   Surface.—Young wood: Smooth. Older wood: Smooth.
Plant foliage: Normal number of leaflets on normal leaves in middle of the stem: 5 to 7 leaflets.
   Leaf size.—80 mm (l)×50 mm (w).
   Quantity.—Average.
   Color.—Mature Foliage: Upper Leaf Surface: Green Group 137A. Lower Leaf Surface: Green Group 137C. Juvenile foliage: Upper Leaf Surface: Green Group 137A. Lower Leaf Surface: Green Group 137C. Anthocyanin intonation: Location: Rachis and petiole. Color: Greyed-Purple Group 185A.
Plant leaves and leaflets:
   Stipules.—Size: 18 mm (l)×5 mm (w). Color: Green Group 137A at Margins. Yellow Green Group 145A at mid-section. Stipitate glands along the leaf margin. Anthocyanin: Color: Greyed-Purple Group 185A.
   Petiole.—Length: 6 to 7 mm. Color: Green Group 137C. Underneath: Stipitate glands observed. Margins: Stipitate glands observed. Anthocyanin: Color: Greyed-Purple Group 185A.
   Rachis.—Color: Green Group 137C. Underneath: Smooth. Margins: Stipitate glands observed. Anthocyanin: Color: Greyed-Purple Group 185A.
   Leaflet.—Edge: Finely serrated. Shape: Ovate. Texture: Moderately glossy and leathery. Arrangement: Odd pinnate. Venation: Reticulate.
Disease resistance: Average resistance to mildew, rust, black spot, and Botrytis under normal growing conditions in Jackson County, Oregon.
Cold hardiness: The variety ‘Poulyc001’ has been found to be cold tolerant to USDA Plant Hardiness Zone 7.

1. A new and distinct climbing rose plant named ‘Poulyc001’ illustrated and described herein due to its abundant Red Group 55C flowers, disease resistance, free branching habit, extended period of bloom and uniform growth.
